(19) United States
(12) Patent Application Publication
(10) Pub. No.: US 2007/0128254 A1
(43) Pub. Date: Jun. 7, 2007

(54) Methods of attaching a dosage form to a medicated tampon assembly

(76) Inventors: Daniel J. Heuer, Fremont, WI (US); Steven C. Gehling, Oshkosh, WI (US); James D. Milner, Appleton, WI (US); Gregory J. Rajala, Neenah, WI (US); Steven R. Burnett, West Chester, OH (US)

Correspondence Address: KIMBERLY-CLARK WORLDWIDE, INC., 401 NORTH LAKE STREET, NEENAH, WI, 54956, US

(21) Appl. No.: 11/297,828

(22) Filed: Dec. 7, 2005

(51) Int. Cl. A61F 13/02; A61K 9/70

(52) U.S. Cl. 424/443; 427/2.31

(57) ABSTRACT

A method for manufacturing a medicated tampon assembly having a tampon body includes positioning a dosage form in a holder, heating the dosage form, contacting the dosage form with the tampon body, and allowing the dosage form to cool. The method may further include positioning a tampon first member in the holder prior to positioning the dosage form in the holder. In addition, a method for manufacturing a medicated tampon assembly having a tampon body includes positioning a dosage form in a holder, placing the dosage form and the tampon body in contact with each other, wherein the tampon body includes fibers, and moving one of the tampon body and the dosage form relative to the other of the tampon body and the dosage form such that the fibers of the tampon body interact with the dosage form.

### BACKGROUND

This invention pertains to methods of manufacturing medicated tampon assemblies used for the application of various therapeutic treatments or preparations into the vaginal or other cavity.

Many disease states and physiological conditions may occur in a woman, including symptoms associated with premenstrual syndrome, menstruation, and menopause. These symptoms may include dysmenorrhea (menstrual cramping), irritability, water retention, moodiness, depression, anxiety, skin changes, headaches, breast tenderness, tension, weight gain, cravings, fatigue hot flashes, itching, and other associated sensory maladies.

Many of these symptoms are due to changes in hormonal levels throughout the menstrual cycle. One example that affects a large number of post-pubescent women is dysmenorrhea, which is the occurrence of painful uterine cramps during menstruation. Menstrual cramping is associated with increased levels of prostaglandin F2α, prostaglandin E2, and, in some cases, leukotrienes in the endometrium and menstrual fluid. These eicosinoids lead to restricted blood flow to the uterus and increased uterine contractions, causing pain.

Various analgesics may be effective in limiting the pain from dysmenorrhea; however some orally-delivered analgesics can cause nausea and vomiting or other untoward side effects; therefore alternative routes of analgesic delivery are of interest.

Attempts have been made to deliver analgesics in the vicinity of the cervix and the vaginal mucosa using various vaginally-inserted devices and methods. Because many of these symptoms typically occur in conjunction with menstruation, some have tried to combine an analgesic with a tampon by coating the tampon, dipping the tampon, or by combining the analgesic with the tampon materials.

For example, in a method of preparation of such a product appropriate for a laboratory setting, a formulation of a fatty compound excipient and an analgesic are heated to a liquid state. Constant mixing of the heated formulation is required to produce a homogeneous formulation. The formulation is then poured onto the tip of a tampon held in a form to contain the liquid. As the formulation cools, the ingredients solidify into a solid waxy substance that has adhered to the absorbent material of the tampon and is thereby securely fastened to the tip of the tampon.

### SUMMARY OF THE INVENTION

Several problems are inherent in a process that attempts to introduce a formulation including a therapeutic agent into or onto a tampon by coating, dipping, solidifying, or the like. Processes such as these may work in a laboratory setting but may not be feasible within an automated tampon manufacturing process. Because of dosing requirements, the formulation including a therapeutic agent must be maintained in a solution that is both homogeneous and of the proper purity to ensure consistent concentration of the therapeutic agent. These requirements are difficult to accomplish during production operation of an automated tampon manufacturing process, and are significantly more difficult to maintain when the automated tampon manufacturing process stops. In addition, different styles and sizes of tampons may have different densities and will absorb an applied liquid formulation including a therapeutic agent differently, resulting in variability in therapeutic agent concentrations across such different tampons.

Specifically, the need to provide constant agitation or mixing of the formulation including a therapeutic agent poses challenges as to how to keep a therapeutic agent homogeneously suspended in a solution when the automated tampon manufacturing process stops. The use of inline mixers and recirculation of the heated liquid formulation during machine stops may provide a method to keep the formulation moving and mixed. However, because a machine could be stopped for several hours, the stability of some formulation mixtures may be compromised by long durations at elevated temperatures, or by mechanical shear forces due to the continuous pumping of the recirculating liquid.

The present invention solves these problems by coupling a dosage form to a tampon to form a medicated tampon assembly. The method allows for a robust mechanical attachment to the tampon and also prevents and/or minimizes absorption of the medication into the tampon during the attaching process. The dosage form, which includes a therapeutic agent, is solid or semi-solid at room temperature, is sufficiently stable, and may be manufactured separately in a controlled facility, whereby dose is easily controlled through controls on concentration and purity.

More specifically, the present invention relates to a method for manufacturing a medicated tampon assembly having a tampon body, the method including positioning a dosage form in a holder, heating the dosage form, placing the dosage form and the tampon body in contact with each other, and allowing the dosage form to cool.

In another aspect, the present invention relates to a method for manufacturing a medicated tampon assembly having a tampon body, the method including positioning a dosage form in a holder, placing the dosage form and the tampon body in contact with each other, wherein the tampon body includes fibers, and moving one of the tampon body and the dosage form relative to the other of the tampon body and the dosage form such that the fibers of the tampon body interact with the dosage form.

In another aspect, the present invention relates to a method for manufacturing a medicated tampon assembly having a tampon body, the method including decreasing the viscosity of at least a portion of a dosage form, contacting a dosage form with the tampon body, and increasing the viscosity of the at least a portion of the dosage form such that the dosage form becomes attached to the tampon body.

In another aspect, the present invention relates to a method for manufacturing a medicated tampon assembly having a tampon body including fibers, the method including positioning a dosage form in a holder, the dosage form having a melting point; contacting the dosage form with the tampon body; heating the dosage form only to a temperature below the melting point of the dosage form by moving the tampon body relative to the dosage form; allowing the dosage form to intermesh with the fibers; and allowing the dosage form to cool.

The present invention relates to a dosage form that is integral with or associated with a feminine care product. The dosage form including the therapeutic agent and excipients may include any therapeutic agent that may be absorbed into the body through the vaginal or other epithelium, or deposited topically on the vaginal or other epithelium, for the purposes of treating a physiological disease, state, or condition.

Other objects and advantages of the present invention will become more apparent to those skilled in the art in view of the following description and the accompanying drawings.

### BRIEF DESCRIPTION OF THE DRAWINGS

FIG. 1 is a view of a two-piece tampon assembly to be used in conjunction with a medicated tampon assembly.

FIG. 2 is a cross-sectional view of the tampon assembly shown in FIG. 1.

FIGS. 3a-3c is a schematic view of a method for manufacturing the medicated tampon assembly of FIG. 1.

FIG. 4a-4d is a schematic view of a method for manufacturing the medicated tampon assembly of FIG. 1.

FIG. 5 is a schematic view of a method for manufacturing the medicated tampon assembly of FIG. 1.

### DETAILED DESCRIPTION OF THE PREFERRED EMBODIMENTS

The invention as described herein will be described for exemplary purposes using a tampon as an example of a feminine care product. The invention, however, applies equally to other forms of products, including tampon-like devices and vaginally-inserted devices, and should not be limited to the example described herein. In addition, although the example described includes a tampon with absorbent material, a product without absorbent material, such as a tampon applicator or other similar applicator, is also contemplated within the invention. Also contemplated is the use of the invention described herein in conjunction with non-catamenial feminine products such as incontinence products, including female incontinence inserts.

The term “surface” and its plural generally refer herein to the outer or the topmost boundary of an object.

The term “dosage form” is used herein as a generic term for a unit form including a formulation that includes a therapeutic agent. The dosage form includes a discrete and consistent quantity of the therapeutic agent to allow for consistent dosing of one receiving the dosage form. The dosage form may be a suppository, a capsule, or any other suitable form. The dosage form may also be spherical, ovoid, domal, generally flat, or any other suitable shape dictated by the needs of the application of the dosage form. The dosage form may have convex, concave, planar, arcuate, or any other suitable surfaces as dictated by the needs of the application of the dosage form.

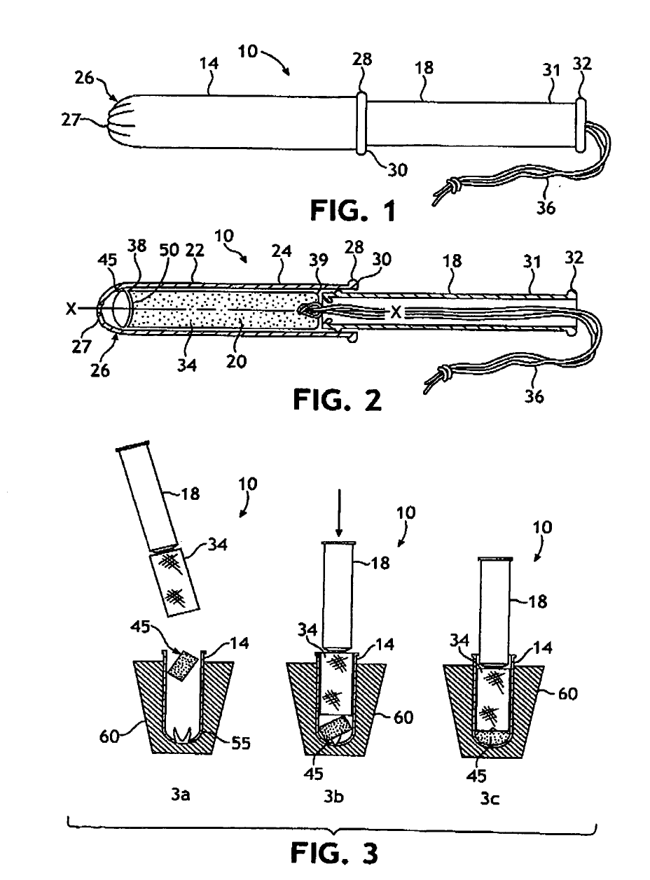

FIGS. 1-2 illustrate a delivery device in the form of a medicated tampon assembly 10, including a first member 14 and a second member 18, which is designed to house a tampon 20 and provide a comfortable means of inserting the tampon 20 into a woman's vagina.

The first member 14 of the medicated tampon assembly 10 may be in the form of a spirally wound, convolutely wound or longitudinally seamed hollow tube which is formed from paper, paperboard, cardboard, plastic, other suitable material, or a combination of such materials. Any plastic in the first member 14 is preferably polyethylene, but may be polypropylene or other suitable plastic. The first member 14, also commonly referred to as an outer tube, may be of any suitable dimensions necessary to house a particular size of tampon 20. The first member 14 has a wall 22 with an outside or exterior surface 24.

The first member 14 is sized and configured to house the tampon 20, and should have a substantially smooth exterior surface 24 which will facilitate insertion of the first member 14 into a woman's vagina. When the exterior surface 24 is smooth and/or slippery, the first member 14 will easily slide into a woman's vagina without subjecting the internal tissues of the vagina to abrasion. The first member 14 may be coated to give it a high slip characteristic. Wax, polyethylene, a combination of wax and polyethylene, cellophane and clay are representative coatings that may be applied to the first member 14 to facilitate comfortable insertion. The first member 14 itself may be formulated to give it a high slip characteristic, including the addition of additives to the resin from which the first member is made, or by an alteration in physical structure of the exterior surface 24, such as adding pebbling or other bumps, to decrease the amount of surface area in contact with the vaginal or other epithelium.

Referring to FIG. 1, an insertion tip 26 is shown having a plurality petals 27 that may radially open such that the insertion tip 26 has a diameter approximately equal to, greater than, or less than the diameter of the first member 14. The petals 27 may be either even or odd in number and may be equally spaced apart or non-uniformly arranged. In another aspect, the insertion tip 26 may be provided without petals 27.

The first member 14 may have a fingergrip ring 28 located proximate the receiving end 30. The fingergrip ring 28 provides an enlarged surface onto which one or more fingers of the user may rest.

As stated above, the medicated tampon assembly 10 includes a second member 18, also commonly referred to as an inner tube. The second member 18, like the first member 14, may be a spirally wound, a convolutely wound or a longitudinally seamed hollow tube constructed from paper, paperboard, cardboard, plastic, other suitable material, or a combination thereof. The second member 18 may be constructed of the same material as the first member 14 or it may be made out of a different material. The second member 18 may also be a solid stick or use some other unique shape. It is also possible to form a finger flange 32 on the free end 31 of the second member 18 to provide an enlarged surface onto which the user's forefinger may rest. The finger flange 32 thereby functions as a seat for the forefinger and facilitates movement of the second member 18 into the first member 14.

In an alternate aspect of the present invention (not shown), the first member 14 and second member 18 together may be replaced by a stick applicator. The stick applicator is used to insert the tampon 20, after which the stick applicator is withdrawn.

A tampon 20 may be an absorbent member primarily designed to be worn by a woman during her menstrual period to absorb menses and other body fluids. The tampon 20 includes a tampon body 34 and a withdrawal string 36. The tampon body 34 is normally compressed into the form of a cylinder and may have a blunt, rounded or shaped forward end. The tampon body 34 has a forward or distal end 38 that is closer to the cervix when the tampon 20 is in use. The tampon body 34 also has a proximal end 39 that is closer to the vaginal opening when the tampon 20 is in use. The tampon 20 commonly has a withdrawal string 36 fastened to the tampon body 34 and extending from the proximal end 39. The withdrawal string 36 serves as a means for withdrawing the tampon 20 from the woman's vagina. Catamenial tampons suitable for use in the present invention include any absorbent material as is known in the art. The tampon body 34 may be formed into specific shapes such as various cup shapes to enhance the therapeutic agent contact area with the cervix, posterior fornix, anterior fornix, lateral fornices, vaginal epithelium areas, or conformance to other anatomical areas within the vaginal or other cavity.

A medicated tampon assembly 10 includes the tampon 20 and also includes a therapeutic agent included in a dosage form 45. For the purposes of this invention, any therapeutic agent that will treat the vaginal or other cavity or will be absorbed into a user's body through the vaginal or other epithelium for the purposes of treating diseases or conditions, promoting the growth of normal vaginal flora, or promoting vaginal health may be used. Examples of therapeutic agents that may be used include, but are not limited to, botanicals, vitamins, moisturizers, antifungal agents, antibacterial agents, pro-biotic agents, calcium, magnesium, hormones, analgesics, prostaglandin inhibitors, prostaglandin synthetase inhibitors, leukotriene receptor antagonists, essential fatty acids, sterols, anti-inflammatory agents, vasodilators, chemotherapeutic agents, and agents to treat infertility.

Some therapeutic agents for use in this invention are absorbable through the vaginal epithelium and travel to the uterus by a unique portal of veins and arteries that are known to exist between the vagina, the cervix, and the uterus. This anastomosis eliminates first-pass metabolism by the liver, effectively delivering higher concentrations of the therapeutic agent to the uterus than would otherwise be available via oral dosing. Those of skill in the art know the efficacy of various therapeutic agents when introduced at a particular anatomical location. For example, when the therapeutic agent is selected to treat dysmenorrhea, it preferably is selected from the following group: nonsteroidal anti-inflammatory drugs (NSAIDs), prostaglandin inhibitors, COX-2 inhibitors, local anesthetics, calcium channel blockers, potassium channel blockers, β-adrenergic agonists, leukotriene blocking agents, smooth muscle inhibitors, and drugs capable of inhibiting dyskinetic muscle contraction.

Alternatively, therapeutic agents modify the vaginal or other environment to enhance the wellness of this anatomical region. The benefits may be rather basic, for example increasing comfort by providing moisturization and/or lubricity. These benefits may also be more complex, for example modulating epithelial cell function to address vaginal atrophy. The beneficial therapeutic agents may reduce negative sensations such as stinging, burning, itching, etc, or introduce positive sensations to improve comfort.

In one aspect of the present invention, the dosage form 45 may be produced in any suitable form including, but not limited to, tablets, capsules, suppositories, gels, disks, lozenges, films, coatings, and other forms. In an alternate aspect of the present invention, the dosage form 45 may be produced in an encapsulated form.

In another aspect of the present invention, the dosage form 45 may be designed to melt at approximately body temperature, or to dissolve or otherwise disperse in the presence of a sufficient aqueous or other liquid trigger, or appropriate chemistry, such as a suitable pH. The dosage form 45 may be in any suitable form including, but not limited to, tablets, capsules, suppositories, disks, lozenges, films, coatings, and other forms.

Additionally, the dosage form 45 may be formed in any shape to promote attachment to the distal end 38 of the tampon body 34 and/or to promote contact with anatomical structures such as the vaginal epithelium, the posterior fornix, the anterior fornix, the lateral fornices, the cervix, or other structures.

The dosage form 45 may include any therapeutic agent, excipient, formulation, compound, or combination thereof that is desirable to introduce into the vaginal or other cavity, including excipients to promote the functionality of the therapeutic agent. The excipients may assist the release of the therapeutic agent, or assist in the absorbency of the therapeutic agent into the vaginal or other epithelium. The use of excipients to facilitate the formulation, delivery, stability, and aesthetic properties of a therapeutic agent delivery system is well known to those familiar with the art.

Examples of materials that may accompany the therapeutic agent in the dosage form 45 include excipients, biologically-compatible adhesives, surfactants, and penetration enhancers. An example of a suitable excipient is SUPPOCIRE suppository base, available from Gattefossé Corp. SUPPOCIRE suppository base is a semi-synthetic glyceride. For example, a suitable semi-synthetic glyceride softens at approximately 36° C. and eventually turns to a liquid as heating the semi-synthetic glyceride decreases its viscosity. At lower temperatures, the semi-synthetic glyceride has a waxy or greasy texture similar to butter or lard. When the semi-synthetic glyceride is heated to just below its melting temperature, the semi-synthetic glyceride takes on a pasty consistency, allowing it to conform to its surroundings. This characteristic allows the semi-synthetic glyceride and thus the dosage form 45 to become mechanically attached to the tampon body 34 without significant absorption into the tampon body 34. In addition, semi-synthetic glycerides such as those that may be included in the dosage form 45 are sensitive to cold working, becoming soft as energy is applied through mechanical shear. Therefore, mechanical means can be used to control temperature using cold flow while dispensing or forming the dosage form 45.

An example of a suitable biologically-compatible adhesive is hydroxypropyl methylcellulose (HPMC), available as METHOCEL* K15M from The Dow Chemical Company. An example of a suitable surfactant is polysorbate 80, available from Spectrum Chemical Manufacturing Corp. An example of a suitable penetration enhancer is LABRAFIL M 1944 C nonionic amphiphilic excipient, available from Gattefossé Corp.

The dosage form 45 may be combined with any absorbent tampon design. The dosage form 45 is preferably positioned at the distal end 38 of the tampon body 34. In alternate aspects of the present invention, the dosage form 45 may be positioned at the proximal or string end 39 of the tampon body 34, or any other suitable position between the distal and proximal ends 38, 39 of the tampon body 34. The dosage form 45 may be designed to partially or fully cover the distal end 38 of the tampon body 34. The tampon body 34 may be formed into specific shapes such as various cup shapes to enhance the therapeutic agent contact area with the cervix, posterior fornix, vaginal or other epithelium areas, or conformance to other anatomical areas within the vaginal or other cavity.

In other various aspects of the present invention, the tampon 20 may include a recess, a dimple, a depression, a concavity, or a reservoir (generically a recess) 50 at the distal end 38 (see FIG. 2), at the proximal end 39, or at any point between the distal and proximal ends 38, 39. The recess 50 is designed to accommodate the dosage form 45. The dosage form 45 may be applied to the recess 50 by any method described herein or by any other suitable method. In an alternate aspect of the present invention, the recess 50 may be formed as a simple dimple. In other alternate aspects of the present invention, the distal end 38 of the tampon 20 may flat, convex, or of any other suitable shape or arrangement.

The dosage form 45 may be produced by the same manufacturer as the manufacturer of the medicated tampon assembly 10. The dosage form 45 may also be produced by a separate manufacturer and provided to the tampon manufacturer in any suitable manner. The tampon 20 and the dosage form 45 may be manufactured at separate facilities and then both shipped to a third party for assembly. As an example, a dosage form manufacturer with a facility specifically designed for pharmaceutical manufacturing that meets current regulatory and quality requirements for drugs and/or devices, as appropriate, may produce the dosage forms 45 under conditions such that homogeneity, concentration, and purity of the dosage form 45 are closely controlled, and such that production is in accordance with applicable regulations. The dosage form 45 may then be sealed and shipped to the manufacturer of the medicated tampon assembly 10. In this manner, the dosage form 45 is produced by a manufacturer with appropriate experience, and the tampon manufacturer may be relieved of establishing a pharmaceutical-production facility. This process is described in more detail in co-pending U.S. patent application Ser. No. 10/335,816 filed on Dec. 31, 2002 and titled “Medicated Tampon.”

The advantages of using a pre-manufactured dosage form over an in-line process where the medicated ingredients are applied to the tampon coincident with the tampon manufacturing process are numerous. The dosage form would be desirably produced at a pharmaceutical manufacturer whose manufacturing facility meets current regulatory and quality requirements for drugs and/or devices as appropriate. This could ensure that a therapeutic agent with the correct dose and purity is dispersed within the dosage form. The use of separately-manufactured dosage forms simplifies the modifications to an existing tampon manufacturing process. The use of separately-manufactured dosage forms allows multiple types of therapeutic agents to be applied to the tampon. The chemical and physical stabilities of the dosage form are not compromised by the assembly process onto the tampon. The process is less dependent on the physical characteristics of the absorbent structure of the tampon, because only a partial phase change of the dosage form may be required to bond with the tampon.

The dosage form 45 may be produced by applying the formulation including a therapeutic agent to a substrate as described in co-pending U.S. patent application Ser. No. 11/090,554 filed on Mar. 25, 2005 and titled “Methods of Manufacturing a Medicated Tampon Assembly.” The dosage form 45 may be coupled to the tampon body 34 by any method described herein.

In a manufacturing aspect of the medicated tampon assembly 10 shown in FIG. 3, an applicator first member 14 is positioned in a mold or holder 60. A dosage form 45 is placed in the first member 14 (see FIG. 3a). A tampon body 34 and optionally a second member 18 are aligned with the first member 14 and are moved into the first member 14, thus pushing the dosage form 45 into the first member 14 (see FIGS. 3b and 3c) at the distal end 38 of the tampon body 34.

In one aspect of the present invention, and referring to FIG. 3, the first member 14 is used as a mold or holder to give the dosage form 45 the shape of the inside of the first member 14 occupied by the dosage form 45. Prior to depositing the dosage form 45 in the first member 14, the petals 27 at the insertion tip 26 of the first member 14 are closed by bringing the petals 27 into contact with a flat or concave shaped surface 55 (see FIG. 3a). The surface may be part of a mold or holder 60. The dosage form 45 is heated to 1°-10° C. below its melting temperature and is deposited into the first member 14. A tampon body 34 is pushed distal end 38 first into the first member 14 until it contacts the dosage form 45 (see FIG. 3b). Pressure from the tampon body 34 causes the dosage form 45 to conform to the shape of the space in the first member 14 occupied by the dosage form 45 (see FIG. 3c). Pressure from the tampon body 34 also causes the dosage form 45 to bond to fibers in the tampon body 34.

In another aspect of the present invention, and referring to FIG. 3, the first member 14 is again used as a mold or holder to give the dosage form 45 the shape of the inside of the first member 14 occupied by the dosage form 45. Prior to depositing the dosage form 45 in the first member 14, the petals 27 at the insertion tip 26 of the first member 14 are closed by bringing the petals 27 into contact with a flat or concave shaped surface 55. The surface may be part of the mold or holder 60. The dosage form 45 is deposited into the first member 14. The dosage form 45 is then heated indirectly by heating one or both of the first member 14 and the tampon body 34. Heating may be accomplished indirectly by heating the mold 60 or directly by heating the first member 14 or the tampon body 34 using electrical heating, by blowing heated gas such as air, or by any other suitable method. The dosage form 45 is heated to 1°-10° C. below its melting temperature. A tampon body 34 is pushed distal end 38 first into the first member 14 until it contacts the dosage form 45. Pressure from the tampon body 34 causes the dosage form 45 to conform to the shape of the space in the first member 14 occupied by the dosage form 45. Pressure from the tampon body 34 also causes the dosage form 45 to bond to fibers in the tampon body 34.

In either aspect, the temperature of the dosage form 45 can also be either controlled directly at the time the dosage form 45 is dispensed into the first member 14 or by controlling the temperature of the tampon body 34 just before the tampon body 34 contacts the dosage form 45 at the time of assembly. In any aspect, heating of the dosage form 45, the tampon body 34, or the first member 14 may be accomplished using heated air, heated liquid, infrared, or any other suitable heating means.

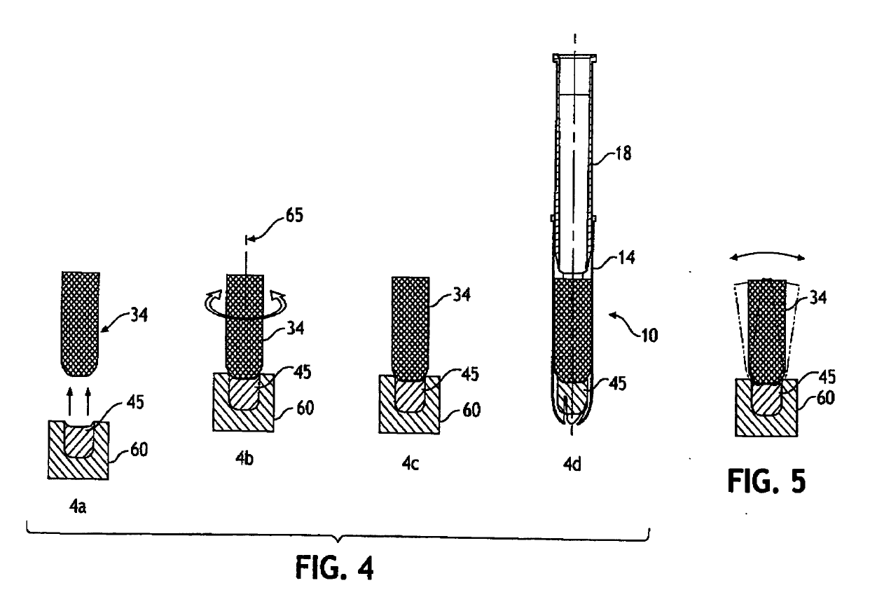

Another aspect of the present invention referred to in FIGS. 4 and 5 takes advantage of the cold working features of the dosage form 45 as described above. A dosage form 45 is placed in a holder 60 at room or other standard temperature (see FIG. 4a). A tampon body 34 is aligned with the dosage form 45 and brought into mild pressing contact with the dosage form 45. The tampon body 34 is then moved relative to the dosage form 45 to generate friction between the tampon body 34 and the dosage form 45. This aspect of the present invention does not require any additional material for bonding or heat that may alter the properties of the tampon. The dosage form 45 may thereby be coupled to the tampon body 34 by entanglement of the fibers of the tampon body 34 with the excipient, by a surface attraction between the excipient and the fibers of the tampon body 34, or by any other suitable mechanism. This coupling may be confirmed by examining the tampon body 34 for the presence of excipient on the fibers.

In one aspect of the present invention shown in FIG. 4b, the dosage form 45 and the tampon body 34 each have a longitudinal axis 65, and at least one of the tampon body 34 and the dosage form 45 is rotated about its longitudinal axis 65, thus generating friction between the tampon body 34 and the dosage form 45. The rotational movement may be continuous (moving in one direction) or oscillatory (alternately moving in different directions). In another aspect of the present invention not shown, one of the tampon body 34 and the dosage form 45 is translated relative to the other, thus generating friction between the tampon body 34 and the dosage form 45. Translational movement may be described as moving one of the tampon body 34 and the dosage form 45 relative to the other such that their longitudinal axes 65 remain at a fixed angle, such as parallel. In another aspect of the present invention shown in FIG. 5, one of the tampon body 34 and the dosage form 45 is rocked relative to the other, thus generating friction between the tampon body 34 and the dosage form 45. Rocking movement may be described as moving one of the tampon body 34 and the dosage form 45 relative to the other such that the longitudinal axis 65 of one changes in angle relative to the longitudinal axis 65 of the other. In another aspect of the present invention, the movement between the tampon body 34 and the dosage form 45 may be a combination of any or all of these aspects. In any of these aspects, the attachment between the tampon body 34 and the dosage form 45 may be enhanced by pre-molding the surface of the dosage form 45 that contacts the tampon body to match the shape of the distal end 38 or other attachment point of the tampon body 34.

Friction between the tampon body 34 and the dosage form 45 creates heat causing semi-synthetic glyceride at the tampon body 34/dosage form 45 interface to soften to the point that the semi-synthetic glyceride wets the tampon fibers. The movement phase lasts from 1.0 to 10.0 seconds depending upon dosage formulation, initial temperature, contact pressure, and movement speed. The movement is ceased and the dosage form 45 is held in contact with the tampon body 34 for an additional 0.1 to 10.0 seconds while the semi-synthetic glyceride at the interface hardens (see FIG. 4c). The dosage form 45 is released from its holder and the tampon body 34 with dosage form 45 now attached is placed inside the first member 14 (see FIG. 4d).

Because a small amount of friction heats the surface of the dosage form 45 enough to soften the surface excipient and allow the tampon fibers on the surface to get coated with the excipient, cold working does work effectively. Even starting with the dosage form 45 at room temperature, relatively little rotation and pressure is required to attach the dosage form 45 to the tampon body 34. One advantage of working with a pre-made dosage form 45 at room temperature is that the dosage form 45 holds its shape and minimizes the chance of tooling contamination due to dosage form residue being left behind in the tooling. Also, working with pre-made dosage forms 45 at room temperature helps ensure that the process of attaching the dosage form 45 to the tampon body 34 does not alter the physical properties of dosage form 45 from the state they were in when the dosage form 45 left its manufacturer.

Because prior work has focused on combining a tampon and an excipient while the excipient was in the liquid state, there has been a focus on using tampons with a highly compressed, radially wound absorbent such as that found in digital tampons. One characteristic of a highly compressed absorbent is that the dense packing creates smaller pores and capillaries than a lightly compressed absorbent. Small pores and capillaries tend to have a quicker liquid uptake but smaller liquid capacity than the same mass of a lightly compressed absorbent. One possibility to explain why highly compressed tampons have been favored previously for drug delivery use has been because the excipient was applied to the tampon while the excipient is in liquid form and the highly compressed tampon absorbs less of the total excipient than a lightly compressed tampon. Thus tampon compression and pre-use absorption was the way prior investigators controlled the drug/tampon interaction.

In one aspect of the present invention, keeping the semi-synthetic glyceride (excipient) just below its melting temperature allows the excipient to bond to the tampon body 34 with minimum absorption into the tampon body 34, no matter the level of tampon compression. Aspects of the present invention are advantageous because liquids need not be dealt with, because molding in place is within the scope of existing machinery technologies, and because a dosage form could be pre-made by a separate facility such as by a pharmaceutical company. In addition, aspects of this invention allow the use of lightly compressed tampons, which exhibit a lower uptake rate than a highly compressed tampon, leaving a body with more therapeutic agent to absorb and more time to absorb it.

In use, and referring to FIG. 2, the medicated tampon assembly 10 functions because the second member 18 is telescopically movable relative to the first member 14. The user may position one or more fingers on the fingergrip ring 28 and one or more fingers on the finger flange 32. The user then squeezes the fingergrip ring 28 and pushes the finger flange 32 toward the fingergrip ring 28 to force the second member 18 further into the first member 14 until the dosage form 45 is expelled from the first member 14. More specifically, as the second member 18 is pushed into the first member 14, the tampon 20 is forced forward against the petals 27. The contact by the tampon 20 causes the petals 27 to radially open to a diameter that is sufficient to allow the tampon 20 to be expelled from the first member 14. With the tampon 20 properly positioned in the vaginal or other cavity, the medicated tampon assembly 10 is withdrawn and properly discarded.

Once the tampon 20 is properly positioned in the vaginal or other cavity, the tampon body 34 absorbs menses and other bodily fluids, and the dosage form 45 delivers the therapeutic agent to the vaginal or other epithelium for local or topical therapeutic action or from there, the therapeutic agent may be transferred to the uterus by normal bodily functions to relieve the condition to be treated.

The invention has been described with reference to various specific and illustrative aspects and techniques. However, it should be understood that many variations and modifications may be made while remaining within the spirit and scope of the invention.

Accordingly, this invention is intended to embrace all such alternatives, modifications and variations that fall within the spirit and scope of the appended claims.

We claim:

1. A method for manufacturing a medicated tampon assembly having a tampon body, the method comprising:
positioning a dosage form in a holder;
heating the dosage form;
placing the dosage form and the tampon body in contact with each other; and
allowing the dosage form to cool.

2. The method of claim 1, further comprising positioning a tampon first member in the holder prior to positioning the dosage form in the holder.

3. The method of claim 2, wherein the dosage form occupies a portion of the tampon first member, and wherein the dosage form is heated sufficiently to take the shape of the portion of the tampon first member occupied by the dosage form.

4. The method of claim 2, wherein heating the dosage form includes heating the dosage form by heating the tampon first member.

5. The method of claim 2, further comprising pushing the tampon body into the first member until it contacts the dosage form.

6. The method of claim 1, wherein heating the dosage form includes heating the dosage form by heating the tampon body.

7. The method of claim 1, wherein heating the dosage form includes moving one of the tampon body and the dosage form relative to the other of the tampon body and the dosage form.

8. The method of claim 7, wherein the moving act includes translational movement.

9. The method of claim 7, wherein the tampon body and the dosage form each have a longitudinal axis, and wherein the moving act includes rotational movement of one of the tampon body and the dosage form about its longitudinal axis.

10. The method of claim 9, wherein the rotational movement is oscillatory.

11. The method of claim 7, wherein the moving act includes rocking one of the tampon body and the dosage form relative to the other of the tampon body and the dosage form.

12. The method of claim 1, wherein the dosage form has a melting point, and wherein the dosage form is heated only to a temperature below the melting point of the dosage form.

13. The method of claim 1, wherein the tampon body includes fibers, and further comprising allowing the dosage form to intermesh with the fibers.

14. The method of claim 1, wherein the tampon body has a distal end, and wherein contacting the dosage form includes contacting the dosage form with the distal end of the tampon body.

15. A method for manufacturing a medicated tampon assembly having a tampon body, the method comprising:
positioning a dosage form in a holder;
placing the dosage form and the tampon body in contact with each other, wherein the tampon body includes fibers; and
moving one of the tampon body and the dosage form relative to the other of the tampon body and the dosage form such that the fibers of the tampon body interact with the dosage form.

16. The method of claim 15, wherein the moving act includes translational movement.

17. The method of claim 15, wherein the tampon body and the dosage form each have a longitudinal axis, and wherein the moving act includes rotational movement of one of the tampon body and the dosage form about its longitudinal axis.

18. The method of claim 17, wherein the rotational movement is oscillatory.

19. The method of claim 15, wherein the moving act includes rocking one of the tampon body and the dosage form relative to the other of the tampon body and the dosage form.

20. A method for manufacturing a medicated tampon assembly having a tampon body, the method comprising:
decreasing the viscosity of at least a portion of a dosage form;
contacting a dosage form with the tampon body; and
increasing the viscosity of the at least a portion of the dosage form such that the dosage form becomes attached to the tampon body.

21. The method of claim 20, wherein decreasing the viscosity includes heating the dosage form.

22. The method of claim 20, wherein increasing the viscosity includes allowing the dosage form to cool.

23. The method of claim 20, wherein the tampon body includes fibers, and further comprising allowing the dosage form to intermesh with the fibers.

24. A method for manufacturing a medicated tampon assembly having a tampon body including fibers, the method comprising:
positioning a dosage form in a holder, the dosage form having a melting point;
contacting the dosage form with the tampon body;
heating the dosage form only to a temperature below the melting point of the dosage form by moving the tampon body relative to the dosage form;
allowing the dosage form to intermesh with the fibers; and
allowing the dosage form to cool.
